(19) United States
(12) Patent Application Publication
(10) Pub. No.: US 2010/0137367 A1
(43) Pub. Date: Jun. 3, 2010

(54) NOVEL CRYSTALLINE BEPOTASTINE METAL SALT HYDRATE, METHOD FOR PREPARING SAME, AND PHARMACEUTICAL COMPOSITION COMPRISING SAME

(75) Inventors: Tae Hee Ha, Hwaseong-si (KR); Chang Hee Park, Yongin-si (KR); Won Jeoung Kim, Suwon-si (KR); Hee Sook Oh, Seoul (KR); Seung Hwan Cho, Busan (KR); Cheol Kyung Kim, Suwon-si (KR); Kwee Hyun Suh, Suwon-si (KR); Gwan Sun Lee, Seoul (KR)

Correspondence Address: SUGHRUE MION, PLLC, 2100 PENNSYLVANIA AVENUE, N.W., SUITE 800, WASHINGTON, DC, 20037, US

(73) Assignee: Hanmi Pharm. Co., Ltd., Hwaseong-si, Gyeonggi-do (KR)

(21) Appl. No.: 12/531,003

(22) PCT Filed: Apr. 4, 2008
(86) PCT No.: PCT/KR2008/001912

§ 371 Date: Sep. 11, 2009

(30) Foreign Application Priority Data
Apr. 5, 2007 (KR) 10-2007-0033756

(51) Int. Cl. A61K 31/4545; C07D 401/12; A61P 17/00

(52) U.S. Cl. 514/318; 546/194

(57) ABSTRACT

The present invention discloses a non-hygroscopic crystalline bepotastine metal salt hydrate, a method for preparing same, and a pharmaceutical composition comprising same for treating or preventing a histamine-mediated disease or an allergic disease.

This is a national stage application under 35 U.S.C. §371 of PCT/KR2008/001912 filed on Apr. 4, 2008, which claims priority from Korean patent application 10-2007-0033756 filed on Apr. 5, 2007, all of which are incorporated herein by reference.

### FIELD OF THE INVENTION

The present invention relates to a crystalline bepotastine metal salt hydrate, a method for preparing same, and a pharmaceutical composition comprising same.

### BACKGROUND OF THE INVENTION

Bepotastine of formula (II), named chemically as (+)-(S)-4-{4-[(4-chlorophenyl)(2-pyridyl)methoxy]piperidino}butyric acid, is a selective fast-acting anti-histaminic agent, which, when orally administered, causes no side effects such as sleepiness and arrhythmia. Bepotastine was originally disclosed as a racemate with the opposite enantiomer (Japanese Laid-open Patent Publication No. Hei 2-25465), but later, bepotastine having S-configuration was known to be pharmacologically much more effective and less toxic than the corresponding R-enantiomer (Japanese Laid-open Patent Publication No. Hei 10-237070).

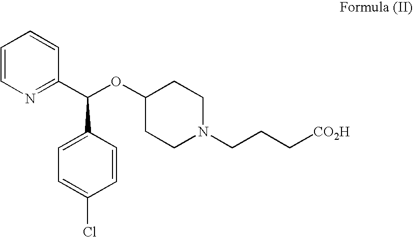

However, bepotastine is obtained in the form of a syrup which is difficult to purify, and due to its high hygroscopic nature, bepotastine can be transformed to R-enantiomer under a moist condition such as the condition encountered during the pharmaceutical formulation and its storage.

Accordingly, there have been attempts to convert bepotastine to an acid salt form having a high optical purity which is resistant to racemization. Japanese Laid-open Patent Publication No. Hei 10-237070 has disclosed bepotastine benzenesulfonic acid salt and bepotastine benzoic acid salt, which are relatively stable and non-hygroscopic. However, it has been found that when bepotastine benzenesulfonic acid salt or bepotastine benzoic acid salt is exposed to a high moisture condition such as 40° C. and 75% relative humidity, it undergoes slow racemization.

Accordingly, the inventors have endeavored to develop a novel form of bepotastine and unexpectedly found that a new crystalline bepotastine metal salt hydrate is non-hygroscopic and chemically or optically stable, and, thus, is effective for preparation of a pharmaceutical bepotastine composition.

### SUMMARY OF THE INVENTION

Therefore, it is an object of the present invention to provide a crystalline bepotastine metal salt hydrate which is non-hygroscopic and highly stable.

It is another object of the present invention to provide a method for preparing the crystalline bepotastine metal salt hydrate.

It is a further object of the present invention to provide a pharmaceutical composition for treating or preventing a histamine-mediated disease or an allergic disease, comprising the crystalline bepotastine metal salt hydrate as an active ingredient.

### BRIEF DESCRIPTION OF THE DRAWING

The above and other objects and features of the present invention will become apparent from the following description of the invention, when taken in conjunction with the accompanying drawings which respectively show:

FIG. 1: an X-ray powder diffraction (XRPD) spectrum of the bepotastine calcium salt hydrate according to the present invention;

FIG. 2: a differential scanning calorimeter (DSC) curve of the bepotastine calcium salt hydrate according to the present invention;

FIG. 3: an XRPD spectrum of the bepotastine strontium salt hydrate according to the present invention;

FIG. 4: a DSC curve of the bepotastine strontium salt hydrate according to the present invention;

FIG. 5: an XRPD spectrum of the bepotastine sodium salt hydrate which is hygroscopic; and

FIG. 6: an XRPD spectrum of the conventional hygroscopic bepotastine potassium salt hydrate.

### DETAILED DESCRIPTION OF THE INVENTION

In accordance with one aspect of the present invention, there is provided a crystalline bepotastine metal salt hydrate of formula (I):

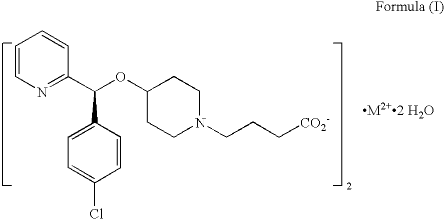

wherein M is calcium or strontium.

The crystalline bepotastine metal salt hydrate according to the present invention is non-hygroscopic and highly stable in terms of maintaining its optical purity.

The bepotastine metal salt hydrate of formula (I) according to the present invention is a crystalline hydrate having two bepotastine molecules coordinated to one calcium ion (II) or one strontium ion (II), to which two H₂O molecules are coordinated. The inventive compound is characterized by its X-ray powder diffraction pattern obtained using CuK_{α} as a lighting source, which shows major peaks at specific 2θ values. Further, the existence of water molecules of the bepotastine metal salt according to the present invention can be confirmed by analyzing its DSC scan, and the number of the water molecules is determined either by thermo-gravity analysis or Karl-Fisher method.

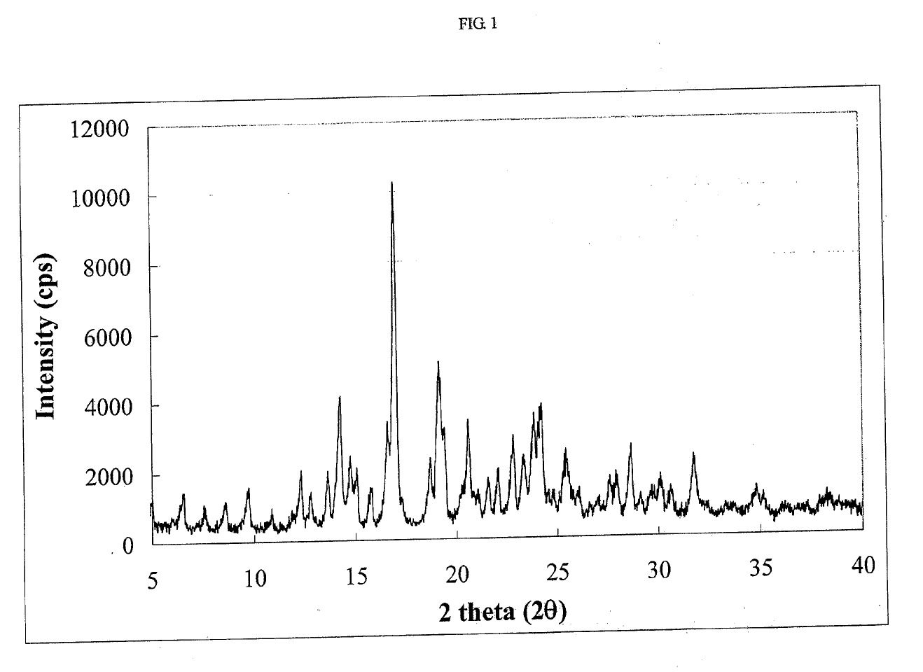

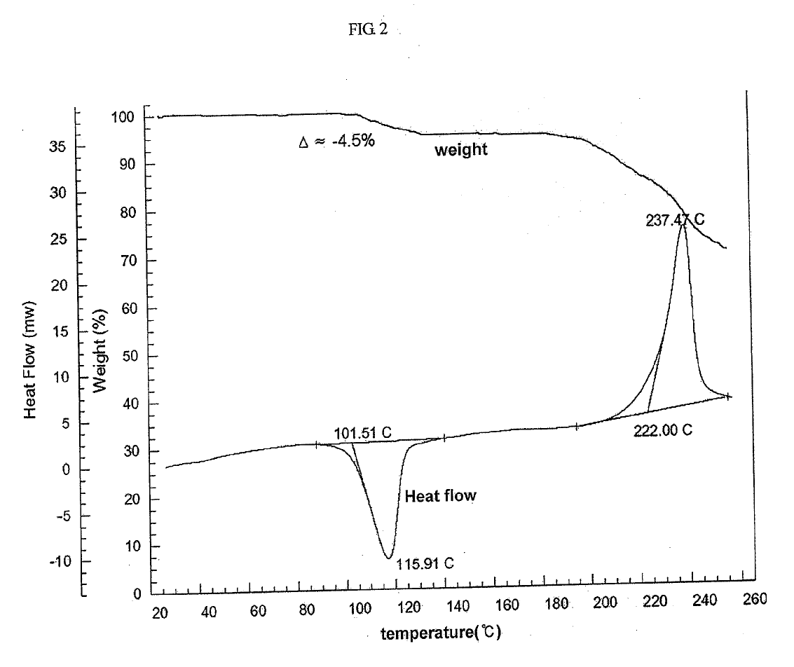

A preferred embodiment of the present invention is the crystalline bepotastine calcium salt dihydrate, whose X-ray powder diffraction (XRPD) spectrum shows peaks having I/Iₒ values of at least 15% (I/I₀×100; I is the intensity of each peak; Iₒ is the intensity of the highest peak) at diffraction angles (2θ±0.2) of 12.3, 14.2, 14.7, 15.1, 16.5, 17.0, 18.7, 19.1, 20.6, 22.8, 23.8, 24.2, 25.5, 28.6 and 31.8 (see FIG. 1). Also, a DSC scan of the inventive bepotastine calcium salt dihydrate shows an endothermic peak at 115.9° C. which corresponds to the dehydrating point, and a thermal weight loss of about 4.5% at the dehydrating point (see FIG. 2). In addition, the water content of the inventive compound determined by Karl-Fisher method is about 4.3% by weight, which is consistent with the theoretical water content of the inventive bepotastine calcium salt dihydrate, i.e. 4.23%.

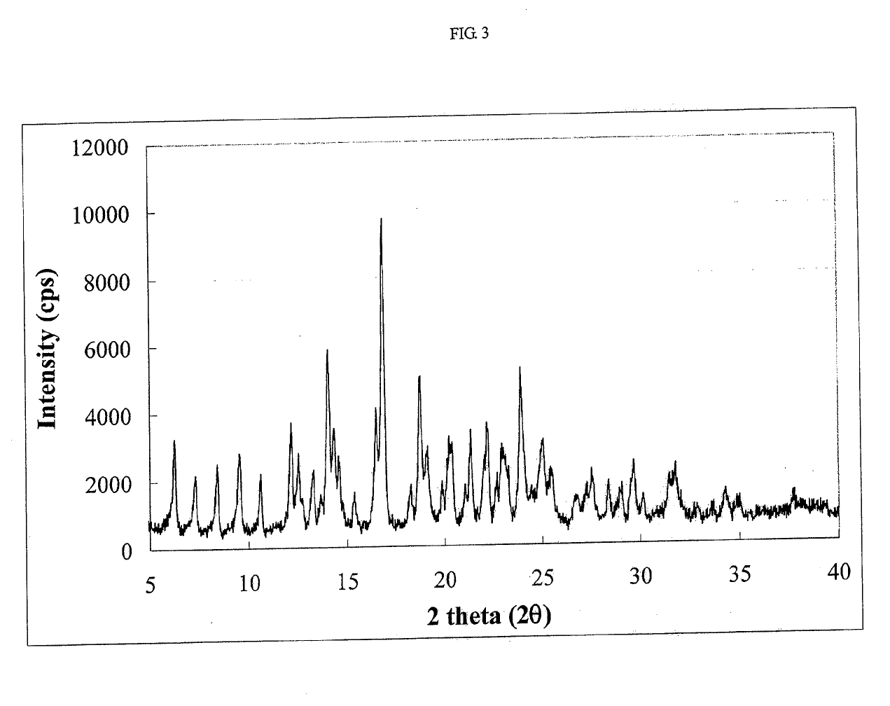

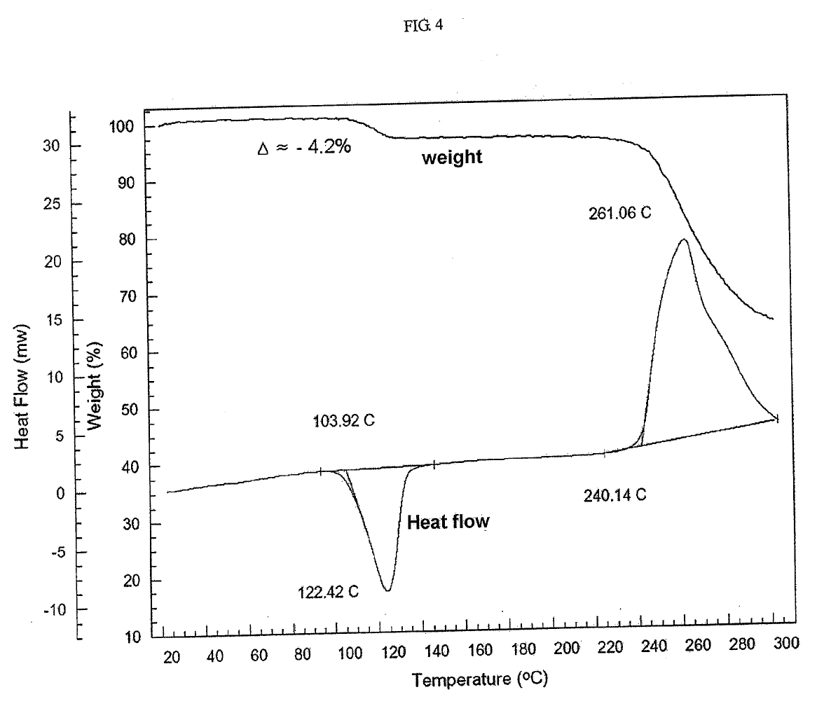

In accordance with another preferred embodiment of the present invention, there is provided the crystalline bepotastine strontium salt dihydrate, whose XRPD spectrum shows peaks having I/Iₒ values of at least 15% at diffraction angles (2θ±0.2) of 4.8, 6.2, 7.3, 8.4, 9.5, 10.6, 12.2, 12.5, 13.3, 14.1, 14.3, 14.6, 16.5, 16.9, 18.7, 19.1, 20.2, 21.3, 22.2, 23.0, 23.9, 25.0, 25.5, 27.5, 29.7 and 31.8 (see FIG. 3). Also, a DSC scan of the inventive bepotastine strontium salt dihydrate shows an endothermic peak at 122.4° C. which corresponds to the dehydrating point, and a thermal weight loss of about 4.2% at the dehydrating point (see FIG. 4). In addition, the water content of the inventive compound determined by Karl-Fisher method is about 4.3% by weight, which is consistent with the theoretical water content of the inventive bepotastine strontium salt dihydrate, i.e. 4.01%.

The optical stability of the crystalline bepotastine metal salt hydrate of the present invention is higher than that of the conventional bepotastine benzenesulfonic acid salt. Accordingly, the crystalline bepotastine metal salt hydrate of the present invention is preferred over the conventional salt in terms of long-term storage stability.

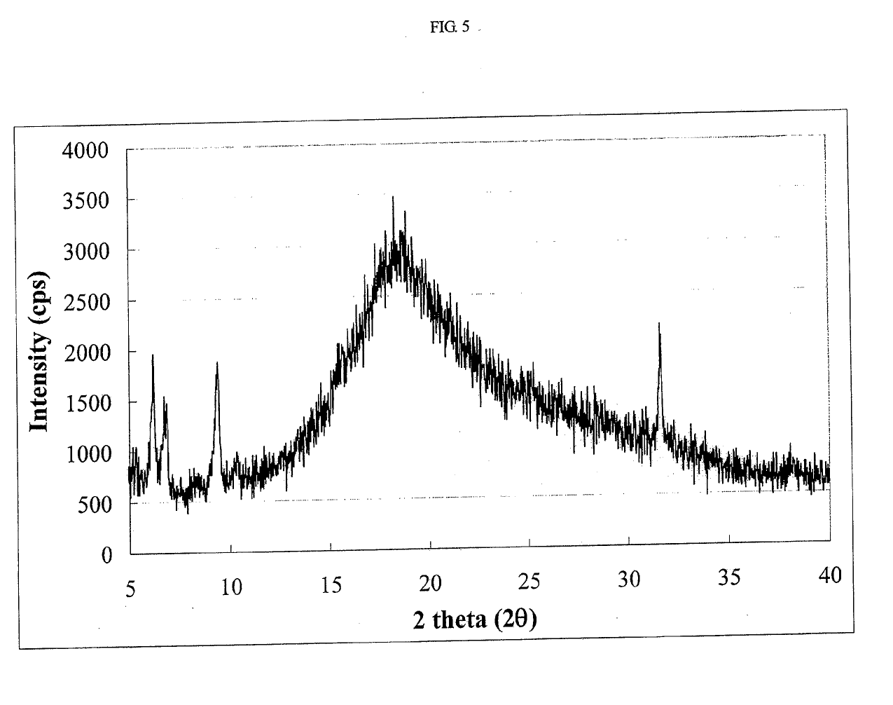

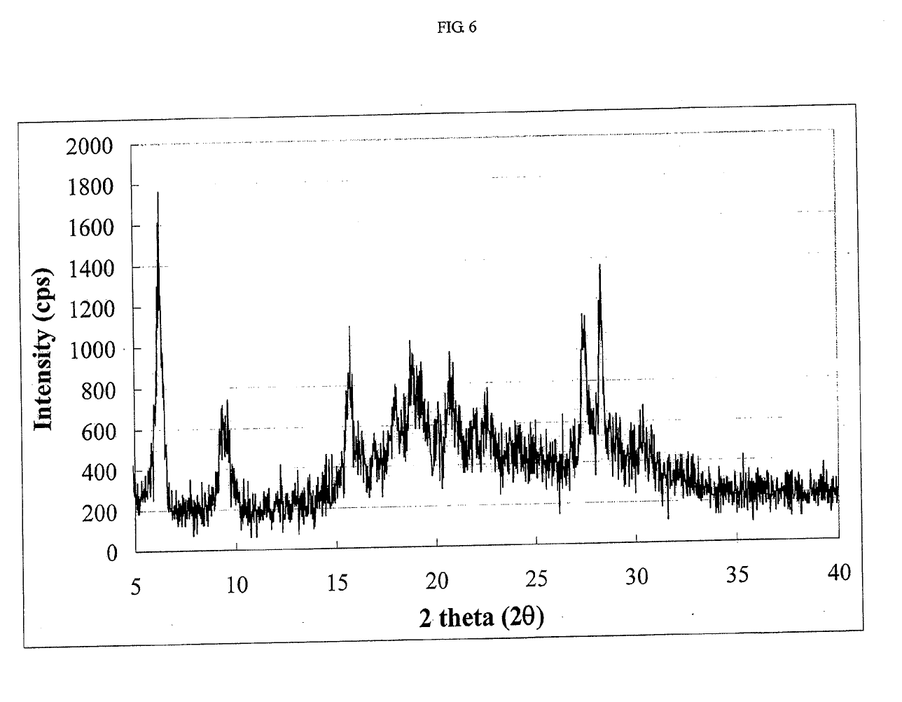

Further, when the crystalline bepotastine metal salt hydrate of formula (I) of the present invention is stored under an extremely high-moisture condition, its moisture content don't increases substantially, thus, it is non-hygroscopic. In contrast, an alkaline metal salt of bepotastine, e.g., lithium salt, sodium salt, potassium salt, magnesium salt or barium salt of bepotastine; a transition metal salt of bepotastine, e.g., zinc salt, aluminum salt, bismuth salt, or iron salt of bepotastine; an organic amine salt of bepotastine, e.g., ammonium salt, ethyl amine salt, dimethyl amine salt, triethylamine salt or N-methyl glucamine salt of bepotastine; and an amino acidic salt of the bepotastine, e.g., arginine salt, lysine salt, or histidine salt of bepotastine, are mostly obtained as non-crystalline forms, and, even when they are obtained as crystalline forms, they are hygroscopic. For example, the sodium salt of bepotastine having a partial crystalline structure is highly hygroscopic, whose XRPD spectrum shows peaks at 2θ diffraction angles of 6.2, 6.8 and 31.6, and a broad peak at 2θ between 15.0 and 25.0 (see FIG. 5); and the potassium salt of bepotastine having a crystalline structure, whose XRPD spectrum shows characteristic peaks at 2θ diffraction angles of 6.3, 9.4, 9.6, 15.7, 18.0, 18.8, 19.3, 20.7, 27.4 and 28.3 (see FIG. 6) is also highly hygroscopic.

TABLE 1

| Metal contained | Diffraction angles (2θ) of | |
|---|---|---|
| in bepotastine salt | X-RPD spectrum | Hygroscopicity |
| Sodium | 6.2, 6.8, 9.4, 15-25(broad), 31.6 | Hygroscopic |
| Potassium | 6.3, 9.4, 15.7, 18.8, 27.4, 28.3 | Hygroscopic |
| Magnesium | Not determined | Hygroscopic |
| Zinc | 11.0, 22.2, 24.7, 28.2, 32.9, 33.5 | Hygroscopic |
| L-Arginine | Amorphous | Hygroscopic |
| L-Lysine | Amorphous | Hygroscopic |

In accordance with the present invention, the crystalline bepotastine metal salt hydrate of formula (I) can be prepared by (i) (a) treating bepotastine with calcium hydroxide or strontium hydroxide in a solvent, or (b) bring bepotastine into contact with a base selected from sodium hydroxide, potassium hydroxide, ammonia and an organic amine in a solvent to obtain a corresponding bepotastine salt, followed by treating said bepotastine salt with a reactive calcium or strontium salt, (ii) inducing the precipitation of crystals from the mixture, and (iii) isolating the precipitated crystals, wherein said solvent used in step (a) or (b) is water or a mixture of water and at least one organic solvent selected from methanol, ethanol, 2-propanol, acetonitrile and acetone.

The solvent employed in the method according to the present invention is used in an amount ranging from 3 to 30 ml, preferably 5 to 15 ml based on 1 g of bepotastine. When a mixture of water and the organic solvent is used, it is preferred that the amount of the organic solvent is not more than 30% by volume based on the total volume of the mixture.

Further, the reacting step (i) is carried out at a temperature ranging from 0° C. to the boiling point of the solvent, preferably from 10 to 50° C., and the precipitating step (ii) is carried out at a temperature ranging from −20 to 50° C., preferably from 0° C. to room temperature.

It is preferred that the amount of calcium hydroxide or strontium hydroxide employed in step (a) is in the range of 0.5 to 0.75 equivalents based on 1 mole of bepotastine.

Further, it is preferred that, in step (b), the base is used in an amount ranging from 1.0 to 1.4 equivalents based on 1 mole of bepotastine, and the reactive calcium or strontium salt, is ranging from 0.5 to 0.75 equivalents based on 1 mole of said base.

Examples of the suitable organic amine are lower organic amines such as methyl amine, dimethyl amine, trimethyl amine, ethyl amine, diethylamine, and triethylamine, and examples of the reactive calcium or strontium salt are halogenated salt, nitric acid salt, sulfuric acid salt, acetic acid salt, oxalic acid salt, or citric acid salt, of calcium or strontium.

As a specific embodiment of the method according to the present invention, bepotastine calcium salt hydrate may be prepared by adding sodium hydroxide to an aqueous solution of bepotastine to obtain a solution containing bepotastine sodium salt, slowly adding a calcium chloride solution thereto, stirring, precipitating, and filtering the precipitated crystal.

Bepotastine employed in the method of the present invention may be prepared in a manner similar to the method disclosed in U.S. Pat. No.6,307,052 or other methods.

The bepotastine metal salt hydrate of formula (I) thus obtained is a non-hygroscopic crystal having a high optical purity of at least 99.5%, and accordingly, it is superior to the commonly used bepotastine benzenesulfonic acid salt in terms of the optical stability. Further, since the high optical purity of the bepotastine metal salt hydrate of formula (I) incorporated in a pharmaceutical composition can be maintained under various conditions such as high humidity and high temperature conditions over a long period of time, the bepotastine metal salt hydrate of the present invention has the added advantage of enhanced storage stability.

Accordingly, the present invention further provides a pharmaceutical composition for treating or preventing a histamine-mediated disease or an allergic disease, which comprises the crystalline bepotastine metal salt hydrate of formula (I) as an active ingredient and a pharmaceutically acceptable carrier.

The pharmaceutical composition according to the present invention is useful for the treating or preventing allergic rhinitis, urticaria, pruritus, nasal obstruction, dermatitis or eczema.

The pharmaceutical composition according to the present invention may be administered via the various routes including oral, nasal, ocular, rectal, and injectable route, preferably the oral route.

For oral administration, the bepotastine metal salt hydrate of the present invention may be formulated with pharmaceutically acceptable carriers, diluents or excipients. Examples of suitable carriers, diluents and excipients are excipients such as starches, sugar and mannitol; filler or extending agents such as calcium phosphate and silica derivatives; binding agents such as cellulose derivatives including carboxymethylcellulose or hydroxypropylcellulose, gelatin, arginic acid salt, and polyvinylpyrrolidone; lubricating agents such as talc, magnesium or calcium stearate, hydrogenated castor oil and solid polyethylene glycol; disintegrants such as povidone, croscarmellose sodium, and crospovidone; and surfactants such as polysorbate, cetyl alcohol and glycerol monostearate. Further, various pharmaceutical composition comprising a specific amount of active ingredient, together with or without additives such as said carriers, diluents or excipients, may be prepared in accordance with any of the conventional procedures (see Remington's Pharmaceutical Science, Mack Publishing Company, Easton, Pa., 19^{th} Edition, 1995).

In a preferred embodiment, the pharmaceutical composition for oral administration of the present invention may contain the crystalline bepotastine metal salt hydrate as an active ingredient in an amount ranging from 0.1 to 95% by weight, preferably 1 to 70% by weight based on the total weight of the composition.

A typical daily dose of the crystalline bepotastine metal salt hydrate of formula (I) for a mammalian including human may range from about 0.5 to 500 mg/kg body weight, preferably 1 to 100 mg/kg body weight, and can be administered in a single dose or in divided doses per one day.

The present invention will be described in further detail with reference to Examples. However, it should be understood that the present invention is not restricted by the specific Examples.

### Optical Purity Analysis

For the measurement of the optical purity of the bepotastine, chiral HPLC was conducted under following conditions and the optical purity was calculated using the Equation 1.

### <Condition>

Detector: Ultraviolet ray spectrophotometer (detection wavelength: 225 nm)
Column: YMC Chiral β-CDs (4.6×250 mm, 5 μm)
Mobile phase: methanol/ammonium acetate buffer solution=45/55(v/v, %)
Flow rate: 0.8 ml/min

Optical Purity (%)=Ps/(Ps+Pr)×100 Equation 1

wherein
Ps is the peak area of bepotastine, and
Pr is the peak area of R-enantiomer of bepotastine.

### Comparative Example 1

### Preparation of Bepotastine Benzenesulfonic Acid Salt

According to the procedure disclosed in U.S. Pat. No. 6,307,052, bepotastine (5.0 g, 12.9 mmol) was dissolved in 250 ml of ethyl acetate, and benzenesulfonic acid monohydrate (2.0 g, 11.4 mmol) was added thereto, followed by concentrating the resulting mixture under a reduced pressure. The residue was dissolved in 250 ml of ethyl acetate and the solution was kept for about 1 week in a refrigerator, to obtain a small quantity of crystals. The crystals were scraped with a specula, and the mixture was kept for another 2 days to induce further precipitation of crystals. The precipitated crystals were collected by filtration, and recrystallized from 50 ml of acetonitrile, to obtain 4.2 g of the title compound (yield: 68%) as an off-white crystalline powder.

Then, bepotastine (45.0 g, 120 mmol) was dissolved in 450 ml of acetonitrile, and bezenesulfonic acid monohydrate (16.1 g, 100 mmol) was added thereto, followed by seeding the resulting mixture with the bepotastine benzenesulfonic acid salt obtained above. The mixture was stirred at room temperature for 12 hours, and the precipitate formed was filtered, to obtain 33 g of the title compound (yield: 66%).
m.p.: 161-163° C. (reference value: 161.5° C.)
water content: 0.4% (Karl-Fisher titrator)
optical purity: 99.9%

The result of X-ray powder diffraction analysis of the above crystalline powder showed peaks having a 100×I/Iₒ value of at least 15% at 2 θ values listed in Table 2.

TABLE 2

| 2θ (±0.2) | d | I/Iₒ |
|---|---|---|
| 12.6 | 9.0 | 19.8 |
| 14.5 | 6.1 | 51.1 |
| 18.4 | 4.8 | 40.2 |
| 19.6 | 4.5 | 100.0 |
| 19.9 | 4.5 | 79.4 |
| 22.2 | 4.0 | 38.8 |
| 24.4 | 3.6 | 30.2 |
| 25.4 | 3.5 | 31.6 |
| 27.4 | 3.3 | 15.0 |

2θ: diffraction angle,
d: distance between crystal faces,
I/Iₒ: relative intensity of the peak.

### Example 1

### Preparation of Bepotastine Calcium Salt Dihydrate

Bepotastine (40.0 g, 100 mmol) was dissolved in 300 ml of water, and sodium hydroxide (4.5 g, 110 mmol) was added thereto, followed by stirring the resulting mixture at room temperature for 30 minutes. Then, calcium chloride (7.3 g, 70 mmol) dissolved in 100 ml of water was slowly added to the mixture, stirred for 12 hours, and the precipitates formed were filtered, to obtain 35 g of the title compound (yield: 83%) as a white crystalline powder.
water content: 4.3% (Karl-Fisher titrator, theoretical value of the dihydrate thereof: 4.23%)
optical purity: 99.9%
m.p.: 236-240° C. (decomposition)

¹H-NMR (DMSO-d⁶, ppm): δ 8.4(d, 1H), 7.8(t, 1H), 7.5(d, 1H), 7.4(m, 4H), 7.2(t, 2H), 5.6(s, 1H), 3.5(m, 1H), 2.6(m, 2H), 2.2(t, 2H), 1.9(m, 4H), 1.8(m, 2H), 1.6(m, 4H).

IR (KBr, cm⁻¹): 3338, 2945, 2825, 1589, 1562, 1490, 1471, 1432, 1412.9, 1308, 1116, 1092, 1061, 1014, 994, 808, 776, 750.

The result of X-ray powder diffraction analysis of the above crystalline powder showed peaks having a 100×I/Iₒ value of at least 15% at 2θ values listed in Table 3.

TABLE 3

| 2θ ± (0.2) | d | I/Iₒ |
|---|---|---|
| 12.3 | 9.2 | 15.5 |
| 14.2 | 6.2 | 38.6 |
| 14.7 | 6.0 | 19.1 |
| 15.1 | 5.9 | 15.1 |
| 16.5 | 5.4 | 24.7 |
| 17.0 | 5.2 | 100.0 |
| 18.7 | 4.7 | 18.0 |
| 19.4 | 4.6 | 45.7 |
| 20.6 | 4.3 | 26.2 |
| 22.8 | 3.9 | 20.0 |
| 23.8 | 3.7 | 25.5 |
| 24.2 | 3.7 | 30.0 |
| 25.5 | 3.5 | 16.5 |
| 28.6 | 3.1 | 18.7 |
| 31.8 | 2.8 | 16.6 |

2θ: diffraction angle,
d: distance between crystal faces,
I/Iₒ: relative intensity of the peak.

### Example 2

### Preparation of Bepotastine Calcium Salt Dihydrate

Bepotastine (8.5 g, 21.9 mmol) was dissolved in a mixture of 60 ml of water and 15 ml of acetone, and sodium hydroxide (0.96 g, 24.0 mmol) was added thereto, followed by stirring the resulting mixture at room temperature for 30 minutes. Then, calcium chloride (1.6 g, 14.4 mmol) dissolved in 25 ml of water was slowly added to the mixture, to obtain a suspension. The suspension was stirred for 12 hours, and the precipitates formed were filtered, to obtain 7.9 g of the title compound (yield: 89%) as a white crystalline powder.
water content: 4.6% (Karl-Fisher titrator)
optical purity: 99.9%
m.p.: 235-239° C. (decomposition)

### Example 3

### Preparation of Bepotastine Calcium Salt Dihydrate

Bepotastine (5.0g, 12.9 mmol) was dissolved in a mixture of 35 ml of water and 2.5 ml of methanol, and sodium hydroxide (0.56 g, 14.0 mmol) was added thereto, followed by stirring the resulting mixture at room temperature for 30 minutes. Then, calcium chloride (0.93 g, 8.4 mmol) dissolved in 12.5 ml of water was slowly added to the mixture, to obtain a suspension. Further, the suspension was stirred for 12 hours, and the precipitates formed were filtered, to obtain 4.1 g of the title compound (yield: 78%) as a white crystalline powder.
water: 4.5% (Karl-Fisher titrator)
optical purity: 99.9%
m.p.: 235-239° C. (decomposition)

### Example 4

### Preparation of Bepotastine Calcium Salt Dihydrate

Bepotastine (5.0g, 12.9 mmol) was dissolved in a mixture of 35 ml of water and 2.5 ml of methanol, and calcium hydroxide (0.56 g, 14.4 mmol) was added thereto, followed by stirring the resulting mixture at room temperature for 12 hours. Next, the precipitate formed were filtered to obtain 4.1 g of the title compound (yield: 78%) as a white crystalline powder.
water content: 4.5% (Karl-Fisher titrator, theoretical value of the dihydrate thereof: 4.23%)
optical purity: 99.9%
m.p.: 235-239° C. (decomposition)

### Example 5

### Preparation of Bepotastine Strontium Salt Dihydrate

Bepotastine (15.0g, 38.6 mmol) was dissolved in 100 ml of water, and sodium hydroxide (1.7 g, 42.5 mmol) was added thereto, followed by stirring the resulting mixture at room temperature for 30 minutes. Then, strontium chloride hexahydrate (3.36 g, 30.3 mmol) dissolved in 50 ml of water was slowly added to the mixture to obtain a suspension. Further, the suspension was stirred for 12 hours and the precipitates formed were filtered, to obtain 15 g of the title compound (yield: 90%) as a white crystalline powder.
water content: 4.3% (Karl-Fisher titrator, theoretical value of the dihydrate thereof: 4.01%)
optical purity: 99.9%
m.p.: 240-245° C. (decomposition)

¹H-NMR (DMSO-d⁶, ppm): δ 8.4(d, 1H), 7.8(t, 1H), 7.5(d, 1H), 7.4(m, 4H), 7.2(t, 2H), 5.6(s, 1H), 3.3(brs, 1H), 2.6(m, 2H), 2.1(t, 2H), 1.9(m, 4H), 1.8(m, 2H), 1.5(m, 4H)

IR (KBr, cm⁻¹): 3332, 2946, 2825, 1589, 1559, 1490, 1471, 1412, 1308, 1114, 1091, 1014, 994, 807, 775, 751.

The result of X-ray powder diffraction analysis of the above crystalline powder showed peaks having a 100×I/Iₒ value of at least 15% at 2 θ values listed in Table 4.

TABLE 4

| 2θ ± (0.2) | d | I/Iₒ |
|---|---|---|
| 4.8 | 18.6 | 25.4 |
| 6.2 | 14.2 | 28.8 |
| 7.3 | 12.2 | 17.8 |
| 8.4 | 10.5 | 20.7 |
| 9.5 | 9.3 | 25.6 |
| 10.6 | 8.3 | 16.2 |
| 12.2 | 7.3 | 30.6 |
| 12.5 | 7.1 | 20.9 |
| 13.3 | 6.7 | 16.3 |
| 14.1 | 6.3 | 55.6 |
| 14.3 | 6.2 | 30.9 |
| 14.6 | 6.1 | 20.1 |
| 16.5 | 5.4 | 35.9 |
| 16.9 | 5.3 | 100.0 |
| 18.7 | 4.7 | 47.6 |
| 19.1 | 4.6 | 23.7 |
| 20.2 | 4.4 | 25.9 |
| 21.3 | 4.2 | 25.0 |
| 22.2 | 4.0 | 29.7 |
| 23.0 | 3.9 | 21.4 |
| 23.9 | 3.7 | 42.9 |
| 25.0 | 3.6 | 24.5 |
| 25.5 | 3.5 | 16.1 |
| 27.5 | 3.2 | 15.2 |
| 29.7 | 3.0 | 17.7 |
| 31.8 | 2.8 | 16.3 |

2θ: diffraction angle,
d: distance between crystal faces,
I/Iₒ: relative intensity of the peak.

### Example 6

### Preparation of Bepotastine Strontium Salt Dihydrate

Bepotastine (5.0g, 12.9 mmol) was dissolved in 25 ml of water, and strontium hydroxide octahydrate (1.89 g, 7.1 mmol) dissolved in 25 ml of water was slowly added thereto, followed by stirring the resulting suspension at room temperature for 12 hours. Then, the precipitates formed were filtered to obtain 4.8 g of the title compound (yield: 86%) as a white crystalline powder.
water content: 4.2% (Karl-Fisher titrator, theoretical value of the dihydrate thereof: 4.01%)
optical purity: 99.9%
m.p.: 230-240° C. (decomposition)

### Experimental Example 1

### Effect of Severe Storage Condition on the Optical Purity

The bepotastine bezensulfonic acid salt obtained in Comparative Example 1 and the bepotastine calcium salt hydrate obtained in Example 1 were respectively exposed to a condition of 60° C. and 75% relative humidity (R.H.) for 4 weeks in either an open or a closed environment. The optical purities of respective bepotastine salts were determined. The results are shown in Table 5.

TABLE 5

| | Optical purity (%)* | | |
|---|---|---|---|
| | | After standing at | |
| Bepotastine | Before | 60° C. and 75% R.H. for 4 weeks | |
| salt | the test | in open condition | in closed condition |
| Benzenesulfonic | 99.9 | 95.5 | 97.3 |
| acid salt | | | |
| Calcium salt | 99.9 | 99.9 | 99.9 |
| hydrate | | | |

As shown in Table 5, the optical purity of the conventional bepotastine benzenesulfonic acid salt decreases significantly during the storage, while the optical purity of the bepotastine calcium salt hydrate according to the present invention remained unchanged.

### Experimental Example 2

### Moisture Absorption Test

The bepotastine bezenesulfonic acid salt obtained in Comparative Example 1 and the bepotastine calcium salt hydrate obtained in Example 1 were respectively kept for 15 days at (i) 25° C. and 75% R.H., (ii) 40° C. and 75% R.H., and (iii) 40° C. and 90% R.H., followed by determining the water contents at day 1, day 3, day 7, and day 15. The results are shown in Table 6.

TABLE 6

| | Temperature, | Water contents (%) | | | | |
|---|---|---|---|---|---|---|
| | Relative | | | | Day | Day |
| Bepotastine salt | humidity | initial | Day 1 | Day 3 | 7 | 15 |
| Benzenesulfonic | 25° C., 75% | 0.4 | 0.6 | 0.6 | 0.7 | 0.6 |
| acid salt | 40° C., 75% | 0.4 | 0.7 | 0.8 | 0.8 | 0.9 |
| | 40° C., 90% | 0.4 | 2.4 | 2.5 | 2.4 | 2.5 |
| Calcium salt | 25° C., 75% | 4.3 | 4.3 | 4.3 | 4.4 | 4.4 |
| hydrate | 40° C., 75% | 4.3 | 4.4 | 4.6 | 4.5 | 4.6 |
| | 40° C., 90% | 4.3 | 4.7 | 4.9 | 4.9 | 5.0 |

As shown in Table 6, no significant change in the water contents was observed for either of the bepotastine salts when stored at 75% R.H. However, at 40° C. and 90% R.H., the water content of the conventional bepotastine benzenesulfonic acid salt increased from 0.4% to 2.5%, whereas that of the inventive bepotastine calcium salt hydrate increased by an increment of less than 0.7%. Therefore, it was confirmed that bepotastine calcium salt hydrate of the present invention is essentially non-hygroscopic.

### Experimental Example 3

### Solubility Test

The saturation solubility of the bepotastine bezenesulfonic acid salt obtained in Comparative Example 1 and the bepotastine calcium salt hydrate of Example 1 were analyzed using pH 1.2 and pH 6.8 buffer solutions. The pH 1.2 and pH 6.8 buffer solution simulate the gastric juice and intestinal juice, respectively. The results are shown in Table 7.

TABLE 7

| | Saturation solubility (mg/ml) | |
|---|---|---|
| Bepotastine salt | pH 1.2 buffer solution | pH 6.8 buffer solution |
| Benzenesulfonic acid salt | 45.0 | 28.1 |
| Calcium salt hydrate | 43.2 | 61.6 |

As shown in Table 7, the solubility of the bepotastine calcium salt hydrate of the present invention at pH 1.2 was similar to that of the bepotastine benzenesulfonic acid salt, whereas, at a pH 6.8 simulating the juice of intestinal region that is responsible to the absorption of bepotastine, the solubility of the bepotastine calcium salt hydrate of the present invention was at least 2 times higher than that of the bepotastine benzenesulfonic acid salt.

Accordingly, the crystalline bepotastine metal salt hydrate of the present invention is non-hygroscopic and optically stable, and, can be stored for long terms without decline of the pharmaceutical activity resulting from the decrease of the optical purity. Therefore, the crystalline bepotastine metal salt hydrate of the present invention is effective as an active ingredient of a pharmaceutical composition for treating or preventing a histamine-mediated disease or an allergic disease.

1. A crystalline bepotastine metal salt hydrate of formula (I): 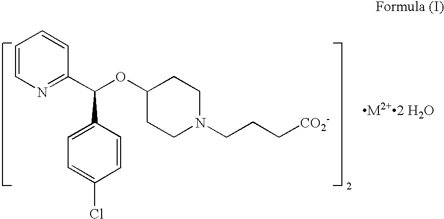
wherein, M is calcium or strontium.

2. The crystalline bepotastine metal salt hydrate of claim 1, wherein M is calcium.

3. The crystalline bepotastine metal salt hydrate of claim 2, whose X-ray powder diffraction spectrum shows peaks having a 100×I/Iₒ value of at least 15% (I is the intensity of each peak; Iₒ is the intensity of the highest peak) at diffraction angles (2θ+0.2) of 12.3, 14.2, 14.7, 15.1, 16.5, 17.0, 18.7, 19.1, 20.6, 22.8, 23.8, 24.2, 25.5, 28.6 and 31.8.

4. The crystalline bepotastine metal salt hydrate of claim 1, wherein M is strontium.

5. The crystalline bepotastine metal salt hydrate of claim 4, whose X-ray powder diffraction spectrum shows peaks having a 100×I/Iₒ value of at least 15% at diffraction angles (2θ+0.2) of 4.8, 6.2, 7.3, 8.4, 9.5, 10.6, 12.2, 12.5, 13.3, 14.1, 14.3, 14.6, 16.5, 16.9, 18.7, 19.1, 20.2, 21.3, 22.2, 23.0, 23.9, 25.0, 25.5, 27.5, 29.7 and 31.8.

6. A method for preparing the crystalline bepotastine metal salt hydrate of claim 1, which comprises
(i) (a) subjecting bepotastine to a reaction with calcium hydroxide or strontium hydroxide in an solvent, or (b) bring bepotastine in contact with a base selected from sodium hydroxide, potassium hydroxide, ammonia and an organic amine in an solvent to obtain a corresponding bepotastine salt, followed by reacting said bepotastine salt with a reactive calcium or strontium salt;
(ii) inducing precipitation of crystals; and
(iii) recovering the precipitated crystals,
wherein the solvent used in step (a) or (b) is water or a mixture of water and at least one organic solvent selected from methanol, ethanol, 2-propanol, acetonitrile and acetone.

7. The method of claim 6, wherein the amount of said calcium hydroxide or strontium hydroxide employed in step (a) is in the range of 0.5 to 0.75 mole equivalent based on the mole of bepotastine employed.

8. The method of claim 6, wherein the amount of said base employed in step (b) is in the range of 1.0 to 1.4 mole equivalent based on the mole of bepotastine employed.

9. The method of claim 6, wherein the amount of said reactive calcium or strontium salt employed in step (b) is in the range of 0.5 to 0.75 mole equivalent based on the mole of base employed.

10. A pharmaceutical composition for treating or preventing a histamine-mediated disease or an allergic disease, comprising the crystalline bepotastine metal salt hydrate of claim 1 as an active ingredient and a pharmaceutically acceptable carrier.

11. The pharmaceutical composition of claim 10, wherein said disease is allergic rhinitis, urticaria, pruritus, nasal obstruction, dermatitis or eczema.

12. The pharmaceutical composition of claim 10, which is one selected from oral, nasal and ocular dosage forms.

13. The pharmaceutical composition of claim 12, which is oral dosage form.

14. The pharmaceutical composition of claim 13, wherein said crystalline bepotastine metal salt hydrate is present in an amount ranging from 0.1 to 95% by weight based on the total weight of the composition.

15. The pharmaceutical composition of claim 14, wherein the amount of said crystalline bepotastine metal salt hydrate is in the range of 1 to 70% by weight based on the total weight of the composition.
